(19) United States
(12) Patent Application Publication
(10) Pub. No.: US 2009/0104296 A1
(43) Pub. Date: Apr. 23, 2009

(54) Discovery of the ability of an effective natural plant product-peeled apple pips (seeds), taken internally-to affect very significantly a human organism delaying the onset of factors associated with ageing

(75) Inventor: Arnold Gilerson, Bronx, NY (US)

Correspondence Address: Arnold Gilerson, Ph.D., Apt 3G, 3871 Sedgwick Ave, Bronx, NY, 10463, US

(73) Assignee: Aleksandr Gilerson, Ph.D., Brooklyn, NY (US)

(21) Appl. No.: 11/998,181

(22) Filed: Nov. 26, 2007

Related U.S. Application Data
(60) Provisional application No. 60/899,501, filed on Feb. 5, 2007.

(51) Int. Cl. A61K 36/73

(52) U.S. Cl. 424/765

(57) ABSTRACT

It was discovered the ability of peeled apple pips (seeds), taken internally:
to affect very significantly a human organism, delaying the onset of factors associated with ageing;
to promote a rejuvenation of the body, a preservation of energy and agility;
to improve a state of body's parts connected with activism of the melanocytes and keratinocytes, including the restoration of one's original hair color, a noticeable decrease of facial wrinkles, a decrease of a tendency for baldness and others;
to cure some illnesses.

### CROSS-REFERENCE TO RELATED APPLICATIONS

Not Applicable

### REFERENCE TO SEQUENCE LISTING

Not Applicable

### BACKGROUND OF THE DISCOVERY

The discovery relates to the field of using of a natural plant product for human body recovering.

As a result of many years of observations and then by specific experiments it was discovered that the apple pips (seeds), taken internally, can influence on some parts and systems of a human body.

It is well-known and described in world science and popular literature that apples are very beneficial for the human body due to their rejuvenating and restorative effects. It is also known that skin of the apples is more effective than the apple's flesh due to its higher concentration of a pectin. The pectin also supports high turgor—an internal pressure—into living cells of the plants and living bodies.

However, the elicited facts show that the rejuvenating effects of apple pips, taken internally, exceed very significantly healthy properties of the apples.

Any information about the influence of the apple pips (seeds), taken internally, on living human body was found neither in literature, nor in the patents.

There is only some information that a composition of some remedies for external application (creams, lotions) contains the apple pips (it was not found any information about the science researches or the patents in this field).

Therefore, it was elicited by this discovery for the first time in science and practice, that apple pips, taken internally, create the series of the effects which rejuvenate and restore of living bodies, and also improve and support noticeable outward aesthetics of some parts of human body by these effects.

This discovery was registered by author—Arnold Gilerson, New York, N.Y.—as U.S. Provisional Patent Application 60/899,501 of Feb. 5, 2007 with title:

“DISCOVERY OF THE ABILITY OF THE PEELED APPLE PIPS (SEEDS), TAKEN INSIDE, TO AFFECT VERY MIGHTLY ON A HUMAN ORGANISM, DELAYING A FORTHCOMING THE FACTORS WHICH CONNECTED WITH ITS AGEING”.

### BRIEF SUMMARY OF THE INVENTION

On a base of many years of observations and facts (over 15 years) then reconfirmed by information about other people, it was discovered for the first time in world science and practice:

The peeled apple pips (seeds), taken internally, generate in the human organism several powerful rejuvenescent and recovering effects that exceed the well-known healthy properties of apples (or their skin).

It was observed that many years' use of apple pips (taken internally):
keeps health in good state,
promotes a significant rejuvenation of the body, a preservation of energy and agility;
influences positively on the body's parts connected with an activism of the melanocytes—hair color, skin's pigmentation;
influences positively on the body's parts (hair, skin, nails) connected with an activism of the keratinocytes—hair follicles and a decrease (as a result) of a tendency for baldness; appearance, growth and overall state of the nails; skin's state and others.

It was discovered that the most immediate changes (occurring in one to two weeks) are:
the restoration of one's original hair color,
a noticeable decrease of the facial wrinkles.

This discovery opens new the widest fields for new researches and new discoveries explaining the phenomenon of rejuvenation and conservation of vital body's ability, possible influence of apple pips substances on immune, cardiovascular, reproductive and other systems of the body.

### DESCRIPTION

### Brief Summary of the Facts, Experiments and Possible Explanations

The influence of apple pips on the restoration one's original hair color was noticed by the author when gray hair first began to appear on the temples and the center of the head (his colleagues by a job and friends of the same age or younger, began to have gray hair and get bald many years earlier).

It was observed that sometimes hair color oscillated between the early stages of graying and getting dark again due to some unknown causes.

Soon it was determined that the restoration of original hair color was connected with the author's habit to eat apple pips following consumption of apple.

This was 15-16 years ago and then these facts were confirmed via many observations. If the author did not consume apples and their pips during one or two months, his gray hair increased. But after a renewal in apple pip consumption, hair color was restored in several days.

As a result, it is the premise of the author to state that his delayed balding and graying can be explained by his apple pips eating habit from a relatively young age (aged approximately 25-30).

In Russia, (prior to the author's resettlement to the United States in 1997) the apples consumed were of Russian and European varieties. Among these were:

Anis, Antonovka, Grushovka moskovskaia, Ranet Krudnera, Slavianka, Jonatan and others.

After moving to the United States, American brands were used for the last 10 years. Among these were:

Golden Delicious, Cortlandt, Fuji, Red Delicious, Granny Smith, McIntosh, Gala, Macoun, Honeycrisp and others.

The following observations showed that apple pips also decrease the wrinkles on women and men's faces. This effect manifests itself relatively quickly, approximately in one to two weeks.

This was confirmed via personal experimentation many times.

Periodical influence of apple pips on the body for a prolonged period results in rejuvenating effects and the preservation of many of the organism's functions including energy, agility, mobility. Many people are incredibly surprised to learn the author's true age based on his physical appearance (relatively wrinkle-free face, hair color, no baldness) and external and internal state of his body (activity, agility, good health, no need to regularly take any prescriptions or medicines). Often they assume he is approximately 15-20 years younger.

The physical appearance of three persons also confirms this conclusion (the author's wife, daughter and grandson, who, following the author's example also consume apple pips). They repeatedly informed the author that the individuals they interacted with often did not believe their true age, generally believing them to be several years younger.

Recently, the role of apple pips and their influence in heart activity was observed: A reduced pulse in the morning (48-50), possibly connected with prescribed medication, during a long period without eating apple pips (6-7 weeks), became more stable after resuming their consumption, increasing to 52-58 pulse beats.

An effect on the skin pigment was observed—various spots of vitiligo on the face and arms became almost imperceptible after regular internal consumption of apple pips. This may be caused by the substances in apple pips affecting the activity of the melanocytes. These melanocytes are responsible for production of melanin (a pigment in hair, skin, and eyes), which determines hair color and skin pigmentation.

It was observed that (in several people) regular consumption of apple pips promote quick growth of the nails, maintain a magnificent appearance, excalation dry cuticles, and ensures nice form, color and luster. This may be caused by the substances in apple pips affecting the activity of the keratinocytes. These keratinocytes are responsible for the production of keratin (“keratin is an extremely strong protein—a major component in skin, hair, nails, and other keratin-containing parts of the body”).

Therefore, it is possible to assume that the chemicals of apple pips actively affect the melanocytes (which give melanin for color of hair and skin) and on the keratinocytes (which form keratin for hair, skin epidermis, nails) and on hair follicle keratinocytes. This serves to explain the delay of baldness.

Side effects were not noticed during many years of regular consumption of apple pips in various quantities.

Later it was discovered that these effects also have place with other persons who are taking internally apple pips.

The author received additional information about several elderly men and women who have a habit of eating apples together with the pips—they have good health and delayed canities and baldness. Some of them noticed periodical restoration of original hair color (unaware of the aforementioned explanation).

The detail information about positive effects of using peeled apple pips are cited in Table 1.

TABLE 1
Confirmations of the efficacy of apple pips' influence on the human body.

| | Persons who take apple pips internally | | | | | |
|---|---|---|---|---|---|---|
| | | | Age | | Term of eating | |
| | | | in | | apples together | |
| Positive effects | Pers. | Sex | 2007 | Place | with pips | Periodicity |

I. Connected with an activism of the melanocytes

| | | | | | | | |
|---|---|---|---|---|---|---|---|
| 1.1. | The restoration | | | | | | |
| | of one's | | | | | | |
| | original hair | | | | | | |
| | color | | | | | | |
| 1.1.1. | | A | M | 82 | Kazan, | All years from | After eating |
| | | (au- | | | Russia; | youth (a habit) | the apples |
| | | thor) | | | New York, | with some pauses | |
| | | | | | USA | (sometimes, | |
| | | | | | (after 1997) | many months | |

Observations: During last 15 years was observed gray hair increases after some intervals
in eating apple pips, and canities decreases after resumption eating pips.
Results: Bigger part of hair has dark color

| | | | | | | |
|---|---|---|---|---|---|---|
| 1.1.2. | F | M | 83 | Kazan, | Many | After eating |
| | | | | Russia | years | the apple (or |
| | | | | | | together) |

Observations: Often gray hair increased and became dark again (the causes were unknown).
Results: Bigger part of hair has dark color

| | | | | | | |
|---|---|---|---|---|---|---|
| 1.1.3. | W | F | 80 | New York, | Approx. | After eating |
| | | | | USA | 5 years | the apples |

Results: White (very gray) hair became almost dark.

| | | | | | | |
|---|---|---|---|---|---|---|
| 1.1.4. | R | F | 82 | Moscow | Many | Together |
| | | | | Russia | years | with eating |
| | | | | | | the apples |
| | | | | | | (or after) |

Results: She does not have gray hair

| | | | | | | |
|---|---|---|---|---|---|---|
| 1.1.5. | N | M | 77 | New York, | Many | Together |
| | | | | USA | years | with eating |
| | | | | | | the apples |

Observations: Many years there are not any changes in quantity of gray hair
Results: Approx. a half of hair are dark.

| | | | | | | | |
|---|---|---|---|---|---|---|---|
| 1.2.1. | The pigmentation's | W | F | 80 | New York, | Approx. 5 | After |
| | changes of skin's | | | | USA | years | eating |
| | spots (vitiligo) on | | | | | | the |
| | a face and arms. | | | | | | apples |

Observation: More regularly eating apple pips last two years.
Results: The spots of vitiligo became almost imperceptible

II Connected with increasing of internal cell's pressure (turgor)

| | | | | | | | |
|---|---|---|---|---|---|---|---|
| 2.1. | A decrease of the | | | | | | |
| | wrinkles on the | | | | | | |
| | men and women's | | | | | | |
| | faces | | | | | | |
| 2.1.1. | | A | M | 82 | New York | Many | After eating |
| | | | | | | years | the apples |
| 2.1.2. | | W | F | 80 | New York | Beginning of | After eating |
| | | | | | | eating apple pips | one apple |
| | | | | | | ~5 years ago | every day |

Observations: In one week the wrinkles decreased on her face (her friends asked her if
she made a cosmetic operation).
Results: A face's state gets better after eating apple pips during 2-3 days and
keeps 2-3 days.

| | | | | | | |
|---|---|---|---|---|---|---|
| 2.1.3. | C | M | 75 | New York | ~One year ago | Together with |
| | | | | | | eating apple |

Observations: The wrinkles were decreasing on the face
Results: No any noticeable wrinkles on his face.

III Connected with an activism of the keratinocytes.

| | | | | | | | |
|---|---|---|---|---|---|---|---|
| 3.1. | The decrease a ten- | | | | | | |
| | dency for baldness - | | | | | | |
| | an influence on | | | | | | |
| | hair follicle | | | | | | |
| | keratinocytes | | | | | | |
| 3.1.1. | | A | M | 82 | Kazan, | See | See |
| | | | | | New York | p.1.1.1. | p.1.1.1. |

Observations: Hair's quantity was decreasing very slowly
Results: No noticeable baldness.

| | | | | | | |
|---|---|---|---|---|---|---|
| 3.1.2. | F | M | 83 | Kazan, | See | See |
| | | | | Russia | p.1.1.2. | p.1.1.2. |

Observations: Hair's quantity was decreasing very slowly.
Results: No noticeable baldness.

| | | | | | | |
|---|---|---|---|---|---|---|
| 3.1.3. | N | M | 77 | New York | See | See |
| | | | | | p.1.1.5. | p.1.1.5. |

Observations: During 5 last years hair's quantity decreasing very slowly (early -
unknown for author).
Results: A little baldness is no increasing during some last years.

| | | | | | | | |
|---|---|---|---|---|---|---|---|
| 3.2. | Permanent magnifi- | | | | | | |
| | cent look of nails, | | | | | | |
| | quick nail's growth, | | | | | | |
| | a lack of dry | | | | | | |
| | cuticles, nice form, | | | | | | |
| | color and luster - | | | | | | |
| | an influence on | | | | | | |
| | nails and skin | | | | | | |
| | keratinocytes | | | | | | |
| 3.2.1. | | W | F | 80 | New York | See p.1.1.3. | See p.1.1.3. |

Observations: One year ago it was observed quick growth of the nails and their magnificent
look.
Results: The magnificent look and quick growth are keeping.

| | | | | | | |
|---|---|---|---|---|---|---|
| 3.2.2. | D | F | 49 | New York | 6 years | Sometimes |
| | | | | | | after eating |
| | | | | | | the apples |

Observations: The same.
Results: The same

| | | | | | | |
|---|---|---|---|---|---|---|
| 3.2.3. | GS | M | 17 | New York | 3 years | After eating |
| | | | | | | the apples |

Observations: The same
Results: The same

| | | | | | | |
|---|---|---|---|---|---|---|
| 3.2.4. | A | M | 82 | New York | See p.1.1.1. | See p.1.1.1. |

Observations: The same
Results: The same

IV Rejuvenation effects of the body

| | | | | | | | |
|---|---|---|---|---|---|---|---|
| 4.1. | Preservation of | A | M | 82 | Kazan, | See | See |
| | energy and agility, | | | | New York | p.1.1.1. | p.1.1.1. |
| | good health | | | | | | |

Observations: Many years is in good mobility and activity. It is not a necessary
in any medicines regularly (an amazing all friends and doctors).
Results: Two years ago main test for the elderly - “maximum breathing capacity”
showed that vital state is equivalent an age 63. Own age feelings fit these estimates.

| | | | | | | | |
|---|---|---|---|---|---|---|---|
| 4.2. | The physical | | | | | | |
| | appearance are | | | | | | |
| | younger than true | | | | | | |
| | age | | | | | | |
| 4.2.1. | | A | M | 82 | Kazan, | See | See |
| | | | | | New York | p.1.1.1. | p.1.1.1. |

Observations: Relatively wrinkle-free face, good hair color, no baldness. Many individuals
interacted with often do not believe in true age (they qualify as 18-20 years younger).
Result: Own feeling and some symptoms of body's state fit younger age.

| | | | | | | |
|---|---|---|---|---|---|---|
| 4.2.2. | W | F | 80 | Kazan, | See | See |
| | | | | New York | p.1.1.3. | p.1.1.3 |

Observations: Many individuals interacted with often do not believe in true age.
Result: Own feelings and some symptoms of body's state fit younger age.

| | | | | | | |
|---|---|---|---|---|---|---|
| 4.2.3. | D | F | 49 | New York | See p.3.2.2. | See p.3.2.2. |

Observation: The same.
Result: The same.

| | | | | | | |
|---|---|---|---|---|---|---|
| 4.2.4. | GS | M | 17 | New York | See p.3.2.3. | See p.3.2.3. |

Observation: The same.
Result: The same.

### DETAILED DESCRIPTION OF APPLYING METHODS

### 1. The Possible Method of the Elaboration of New Fast Acting Effective Product (Taken Internally) for the Restoration of One's Original Hair Color

1.1. The Method Used by the Author (Initial Products, Quantity, Time Intervals).

Ingestion of pips from one apple (one apple has 10 pips or less) and then from 2-3 apples (up to 20-25) every day—as a rule, it was after eating one apple—the newly grayed hair darkened in one to two days. Continued ingestion of pips during several days intensifies the darkening of hair and preserves them in that state.

An omission of pips from the diet for several weeks causes an increase of gray hair. If the pip consumption is resumed once more, the process of hair color restoration will resume as well. The hair on other parts of body become darker as well, particularly evident near the wrist.

It is therefore possible to suppose (via author's experience) that regular consumption of apple pips at the approximate origin graying, will effectively postpone it for many years.

It was observed (about 3-4 years ago) that the different kinds of apple have a different efficiency for restoring hair color—“Golden Delicious” apples are better then others.

1.2. The Possible Method of an Elaboration for the Manufactory New Effective Product.

It is expediently more thorough detections 2-3 apple kinds, the pips of which give maximum efficacy for hair color restoration.

The research can be made by comparative laboratory analyses of chemical composition, enzymatic and other substances of “strong” and “weak” apple kinds (for example, Golden Delicious and Red Delicious), and then by determining the differences in their effect of the hormones and activity of the melanocytes in the follicles which define canities (hair graying). Other researches may be required as well.

After selecting the most effective apple type, it is necessary to acquire the tests for hair color restoration (with the men and women's groups):
in beginning of graying
in 8-12 months after graying
in 2-3 years after graying
in 5-6 years after graying
   The experiment will have to be conducted as it was explicated in 1.1 (may be with the necessary corrections).

It is necessary to remember that the pips were consumed (as a rule) after eating one apple. Therefore, it is necessary to conduct the tests in two versions: a) after eating one apple and b) without eating an apple.

Through this research any and all possible side effects, provided they are present, will be determined.

If all tests prove to be in compliance with the data, a new fast acting product can be produced shortly thereafter (because there are the machines for pip's peeling and pulverizing)

1.3. The Convenient Version for Sale.

If it is determined that the apple pips are more effective with an apple, one can use a convenient version for sale.

Each portion of pips can be mixed with some natural apple product, just about as one apple for one dose of the pips. It can be, for example, 100 gram of an apple sauce.

### 2. The Elaboration of New Fast Acting Effective Product (Taken Internally) to Decrease Wrinkles

2.1. The Used Method.

The apple pips used of 1 or 2-3 apples (10-15-25 pips), after eating one apple. After 4-5 taking pips (during one week), the wrinkles were decreasing noticeably on the face an elderly woman (her friends asked her if she made a cosmetic operation). That wrinkles decreasing was continuing during 4-5 days. After next 2-3 takings, noticeable effect was evincing in 1-2 days.

2.2. The Possible Method for Manufacturing of a New Product

First check-up can be made easily in accordance with 2.1.

The particular researches and practical experiments and also marketing research must show a practicability of creation one product by p.p.1 and 2 or it is better to have two different products.

The experiences showed that the restoration and preservation of hair color need more long preliminary and regular uses of product. The effect of wrinkles decrease evinces sooner with using more portions of the product (in 1-2 days). But both effects evince in both events.

Any side effects were not noticed during many years regular uses of apple pips (up to 25-30 pips for one dose).

### 3. The Possible Organization of Works for Further Researches of Discovered Rejuvenating and Recovering Effects Connected with Influence Apple Pips on the Human Body and for Creation New Rejuvenating Remedies and Drugs

3.1 The Discovery Significant Positive Affects which are Connecting with Using Per Os Apple Pips, on Activity of the Melanocytes and Keratinocytes Determines Next Possible Pathways of the Researches:

Hair Color

Elaboration of the remedies for:
delaying of canities
delaying and treatment of an albinism and other abnormal manifestations connecting with hair pigmentation

Hair State

Elaboration of the remedies for:
delaying of baldness
treatment alopecia and other hair diseases
against dandruff

Skin Pigmentation

Elaboration of the remedies:
for treatment vitiligo
against the freckles
for treatment other diseases connected with skin pigmentation—albinism, melasma and others

Skin Diseases

The researches and elaboration of the remedies for treatment such serious illnesses as psorias, melanomas and others.

Nails State

Elaboration of the remedies for:
supporting nice appearance of the nails
treatment different diseases finger and toes nails.

Elaboration of the remedies for treatment other diseases of hair, skin, nails, eyes, mouth and other parts of body, connected with activity of the melanocytes and keratinocytes.

3.2. The Researches by p.1. 2. have to Elicit the Main Active Substances, Containing into Apple Pips, that Influence Rejuvenation and Recovering Processes.

In new researches:
it can be verified an influence increase of concentration those active substances to more fast development of positive effects;
it is possibly that it will be discovered natural or synthesized products or catalysts for increasing positive effects (the author discovered one of them for hair color restoration).

3.3. New Perspective Directions are Opening for the Researches of the Influences Apple Pips on the Body's State:

Influences on:
cardiovascular system
immune system
reproductive system and others.

Undoubtedly, there are many other factors affecting to recovering and rejuvenation of human organism by apple pips substances. It is the widest field of the researches.

A realization these special, laboratory and clinical researches with using new created products, can open new significant recovering effects.

It also needs to take into consideration that all new products will possess by all well-known useful qualities of pectin—inclusive products with their maximum efficacy, and with upholding high turgor in the cells of the body.

I claim:

1. It was discovered a new, previously unknown to world science and practice the ability of peeled apple pips, taken internally, to affect very significantly the human organism, delaying the onset of factors associated with its ageing, and also to create some medicinal actions.

2. A method according to claim 1. for the restoration of one's original hair color.

3. A possible method, in accordance with claims 1 and 2, for the manufacture and sale of a new product.

4. A method according to claim 1. for a decreasing the wrinkles on men and women's faces.

5. Any products or the drugs, in accordance with claim 1, for rejuvenation, treatment and restoration any organisms, including preservation of the activity, the decrease a tendency to the baldness and other effects.

6. Any products or medicines, in accordance with claim 1. for alteration or treatment any skin pigmentation (as vitiligo and others) or other processes (skin diseases) associated with activity of the melanocytes.

7. Any products or drugs, in accordance with claim 1. for alteration or treatment any processes or diseases in hair, skin, nails and other protein containing parts of body associated with activity of the keratinocytes.
